SEQUENCE LISTING (44 sequences)

SEQ ID NO: 1 - protein, 12 aa, Artificial Sequence
  Synthetic Polypeptide
XXXXPAAXXXXX

SEQ ID NO: 2 - protein, 4 aa, Artificial Sequence
  Synthetic Polypeptide
PAAG

SEQ ID NO: 3 - protein, 8 aa, Artificial Sequence
  Synthetic Polypeptide
GAPAAGAP

SEQ ID NO: 4 - protein, 11 aa, Artificial Sequence
  Synthetic Polypeptide
DAGAPAAGPVV

SEQ ID NO: 5 - protein, 11 aa, Artificial Sequence
  Synthetic Polypeptide
DAGAPAAGAPV

SEQ ID NO: 6 - protein, 7 aa, Artificial Sequence
  Synthetic Polypeptide
AGAPAAK

SEQ ID NO: 7 - protein, 13 aa, Artificial Sequence
  Synthetic Polypeptide
DAPAAPAGPAVPV

SEQ ID NO: 8 - protein, 11 aa, Artificial Sequence
  Synthetic Polypeptide
DAAAPAGAPVV

SEQ ID NO: 9 - protein, 10 aa, Artificial Sequence
  Synthetic Polypeptide
DAPAAPAVPV

SEQ ID NO: 10 - protein, 11 aa, Artificial Sequence
  Synthetic Polypeptide
DPGAAAAGAPV

SEQ ID NO: 11 - protein, 11 aa, Artificial Sequence
  Synthetic Polypeptide
DPGAAPAGAPV

SEQ ID NO: 12 - protein, 11 aa, Artificial Sequence
  Synthetic Polypeptide
DAPAAAAGAPV

SEQ ID NO: 13 - protein, 11 aa, Artificial Sequence
  Synthetic Polypeptide
DAPAAPAGAPV

SEQ ID NO: 14 - protein, 11 aa, Artificial Sequence
  Synthetic Polypeptide
DAGPAAAGAPV

SEQ ID NO: 15 - protein, 11 aa, Artificial Sequence
  Synthetic Polypeptide
DAGAAPAGAPV

SEQ ID NO: 16 - protein, 11 aa, Artificial Sequence
  Synthetic Polypeptide
DAGAPAPGAPV

SEQ ID NO: 17 - protein, 11 aa, Artificial Sequence
  Synthetic Polypeptide
DAGAPAAPAPV

SEQ ID NO: 18 - protein, 7 aa, Artificial Sequence
  Synthetic Polypeptide
EAAPAGA

SEQ ID NO: 19 - protein, 11 aa, Artificial Sequence
  Synthetic Polypeptide
DAAPAGAGAPV

SEQ ID NO: 20 - protein, 9 aa, Artificial Sequence
  Synthetic Polypeptide
DAAPAGAPV

SEQ ID NO: 21 - protein, 8 aa, Artificial Sequence
  Synthetic Polypeptide
DPAAPAGA

SEQ ID NO: 22 - protein, 7 aa, Artificial Sequence
  Synthetic Polypeptide
AAPAGAD

SEQ ID NO: 23 - protein, 7 aa, Artificial Sequence
  Synthetic Polypeptide
AAPAGAK

SEQ ID NO: 24 - protein, 7 aa, Artificial Sequence
  Synthetic Polypeptide
KAAPAGA

SEQ ID NO: 25 - protein, 9 aa, Artificial Sequence
  Synthetic Polypeptide
EAAPAGAPV

SEQ ID NO: 26 - protein, 9 aa, Artificial Sequence
  Synthetic Polypeptide
DAAPAAPVV

SEQ ID NO: 27 - protein, 8 aa, Artificial Sequence
  Synthetic Polypeptide
DAPAAPVV

SEQ ID NO: 28 - protein, 7 aa, Artificial Sequence
  Synthetic Polypeptide
DAGAPAA

SEQ ID NO: 29 - protein, 6 aa, Artificial Sequence
  Synthetic Polypeptide
DAGAPA

SEQ ID NO: 30 - protein, 5 aa, Artificial Sequence
  Synthetic Polypeptide
DAGAP

SEQ ID NO: 31 - protein, 6 aa, Artificial Sequence
  Synthetic Polypeptide
AAGAPV

SEQ ID NO: 32 - protein, 5 aa, Artificial Sequence
  Synthetic Polypeptide
AGAPV

SEQ ID NO: 33 - protein, 4 aa, Artificial Sequence
  Synthetic Polypeptide
GAPV

SEQ ID NO: 34 - protein, 7 aa, Artificial Sequence
  Synthetic Polypeptide
DAGAPAA

SEQ ID NO: 35 - protein, 9 aa, Artificial Sequence
  Synthetic Polypeptide
DAGAPAAPV

SEQ ID NO: 36 - protein, 7 aa, Artificial Sequence
  Synthetic Polypeptide
EAGAPAA

SEQ ID NO: 37 - protein, 9 aa, Artificial Sequence
  Synthetic Polypeptide
EAGAPAAPV

SEQ ID NO: 38 - protein, 7 aa, Artificial Sequence
  Synthetic Polypeptide
KAGAPAA

SEQ ID NO: 39 - protein, 8 aa, Artificial Sequence
  Synthetic Polypeptide
DPAGAPAA

SEQ ID NO: 40 - protein, 6 aa, Artificial Sequence
  Synthetic Polypeptide
AAPAGA

SEQ ID NO: 41 - protein, 4 aa, Artificial Sequence
  Synthetic Polypeptide
APAA

SEQ ID NO: 42 - protein, 7 aa, Artificial Sequence
  Synthetic Polypeptide
DAAPAGA

SEQ ID NO: 43 - protein, 5 aa, Artificial Sequence
  Synthetic Polypeptide
LPFFD

SEQ ID NO: 44 - protein, 13 aa, Artificial Sequence
  Synthetic Polypeptide
GAAAAGAVVGGLG

(19) United States
(12) Patent Application Publication
(10) Pub. No.: US 2005/0181998 A1
(43) Pub. Date: Aug. 18, 2005

(54) Prion inhibiting peptides and derivatives thereof

(75) Inventors: Celine Adessi, St Louis (FR); Serge Halazy, Monthoux (FR); Gabriela Saborio, Ferney-Voltaire (FR); Claudio Soto-Jara, Galveston, TX (US)

Correspondence Address: OBLON, SPIVAK, MCCLELLAND, MAIER & NEUSTADT, P.C., 1940 DUKE STREET, ALEXANDRIA, VA, 22314, US

(73) Assignee: APPLIED RESEARCH SYSTEMS ARS HOLDING N.V., Curacao (AN)

(21) Appl. No.: 10/497,160

(22) PCT Filed: Dec. 9, 2002
(86) PCT No.: PCT/EP02/13915

(30) Foreign Application Priority Data
Dec. 10, 2001 (EP) 01000733.4

(51) Int. Cl. A61K 38/08; C07K 7/08; C07K 7/06

(52) U.S. Cl. 514/16; 514/17; 530/328; 530/329; 530/330

(57) ABSTRACT

Short peptides and derivatives or analogs thereof for the treatment or prevention of transmissible spongiform encephalopathies, in particular CJD are herein described. These peptides and/or their derivatives have been designed to block the conformational changes that occur in the prion protein (PrP) and which are implicated in the pathogenesis of transmissible spongiform encephalopathies as well as to dissolve the fibrillar deposits already formed

### FIELD OF THE INVENTION

Novel short peptides and derivatives or analogs thereof for the treatment or prevention of transmissible spongiform encephalopathies, in particular CJD. These peptides and/or their derivatives have been designed to block the conformational changes that occur in the prion protein (PrP) and which are implicated in the pathogenesis of transmissible spongiform encephalopathies as well as to dissolve the fibrillar deposits already formed.

### BACKGROUND OF THE INVENTION

Transmissible spongiform encephalopathies (TSE) also known as prion diseases are a group of neurodegenerative diseases that affect humans and animals. Creutzfeldt-Jakob disease (CJD), kuru, Gerstmann-Straussler-Scheiker disease (GSS) and fatal familial insomnia (FFI) in humans as well as scrapie and bovine spongiform encephalopathy (BSE) in animals are some of the TSE diseases (Prusiner, 1991).

Although these diseases are relatively rare in humans, the risk for the transmissibility of BSE to humans through the food chain has taken the attention of the public health authorities and the scientific community (Cousens et al., 1997, Bruce et al., 1997).

These diseases are characterized by an extremely long incubation period, followed by a brief and invariably fatal clinical disease (Roos et al., 1973). To date no therapy is available.

The hallmark event of the disease is the formation of an abnormally shaped protein named PrP^{Sc}, which is a post-translationally modified version of a normal protein, termed PrP^{C} (Cohen and Prusiner, 1998). Chemical differences have not been detected to distinguish both PrP isoforms (Stahl et al., 1993) and the conversion seems to involve a conformational change whereby the α-helical content of the normal protein diminishes and the amount of β-sheet increases (Pan et al., 1993). The structural changes are followed by alterations in the biochemical properties: PrP^{C} is soluble in non-denaturing detergents, PrP^{Sc} is insoluble; PrP^{C} is readily digested by proteases, while PrP^{Sc} is partially resistant, resulting in the formation of a N-terminally truncated fragment known as PrPres (Baldwin et al., 1995, Cohen and Prusiner, 1998).

Prion replication is hypothesized to occur when PrP^{Sc} in the infecting inoculum interacts specifically with host PrP^{C}, catalyzing its conversion to the pathogenic form of the protein (Cohen et al., 1994). This process takes from many months to years to reach a concentration of PrP^{Sc} enough to trigger the clinical symptoms.

β-sheet breaker peptides have so far been designed to block the conformational changes that occur in both Aβ and prion protein (PrP), which are implicated in the pathogenesis of Alzheimer's and prion disease, respectively. The prior art has previously shown that 11- and 5-residue β-sheet breaker peptides (namely, iAβ1 and iAβ5, respectively) homologous to the central hydrophobic region of Aβ, inhibit peptide conformational changes that result in amyloid formation and also dissolve preformed fibrils in vitro (see WO 96/39834). In addition, the 5-residue peptide is capable of preventing the neuronal death induced by the formation of β-sheet rich oligomeric Aβ structures in cell culture experiments. Furthermore, by using a rat model of amyloidosis induced by intracerebral injection of Aβ 1-42, the prior art has shown that co-injections of the 5-residue β-sheet breaker peptide decreased cerebral Aβ accumulation and completely blocked the deposition of fibrillar amyloid-like lesions in the rat brain. Finally, the β-sheet breaker peptide injected eight days after the injection of Aβ was able to disassemble preformed Aβ fibrils in the rat brain in vivo, that leads to a reduction in the size of amyloid deposits. Interestingly, removal of amyloid by the β-sheet breaker peptide reverts the associated cerebral histologic damage, including neuronal shrinkage and microglial activation (Soto et al., 1996 and Soto et al., 1998)

β-sheet breaker peptides have also been designed to prevent and to reverse conformational changes caused by prions (PrP). Based on the same principles and using as a template the PrP sequence 115-122, the prior art has shown that when a set of β-sheet breaker peptides was synthesized, a 13-residue peptide (iPrP13, SEQ ID NO: 44) showed the greatest activity. Several in vitro cell culture and in vivo assays were used to test for inhibitory activity and the results clearly indicated that it is possible not only to prevent the PrP^{C}->Prp^{Sc} conversion, but more interestingly to reverse the infectious PrP^{Sc} conformer to a biochemical and structural state similar to PrP^{C} (Soto et al., 2000).

Short peptides have been utilized extensively as drugs in medicine.

However, the development of peptide drugs is strongly limited by their lack of oral bioavailability and their short duration of action resulting from enzymatic degradation in vivo. Progress in recent years toward the production of peptide analogs (such as pseudopeptides and peptide mimetics) with lower susceptibility to proteolysis has increased the probability to obtain useful drugs structurally related to their parent peptides. Improving peptide stability to proteases not only increases the half-life of the compound in the circulation but also enhances its ability to be transported or absorbed at different levels, including intestinal absorption and blood-brain barrier permeability, because transport and absorption appear to be highly dependent upon the time of exposure of membranes or barriers to the bioactive species.

WO 01/34631 reports some derivatives of known β-sheet breaker peptides, which derivatives show an improved half-life and an increased biological activity with respect to the corresponding non-derivatized peptides.

### DESCRIPTION OF THE INVENTION

We have designed compounds that reverse the PrP^{Sc} structure and properties based on the hypothesis that the formation of the pathological protein can be inhibited and reversed by synthetic peptides homologous to the PrP regions implicated in the abnormal folding, but modified to contain specific β-sheet blocker residues (β-sheet breaker peptides). We aimed to generate synthetic peptides shorter than the β-amino acids prion-inhibitor peptide already disclosed. These short peptides would specifically interact with PrP because of their sequence homology, and would induce unfolding of β-pleated sheet structure because of their inability to adopt the β-sheet conformation. The PrP region selected for designing β-sheet breaker peptides corresponds to the conserved region spanning residues 115-122 (SEQ ID NO: 44) of PrP (FIG. 1), since some evidence suggests that this region has a central role in the conversion of PrP^{C} to PrP^{Sc}. Prolines were used as β-sheet blockers since the occurrence of this residue in a β-pleated structure is energetically unfavourable because of the constraints on its ability to support the required peptide backbone conformation. According to these principles, a set of about 50 putative prion inhibitors was designed (FIG. 2) and tested. On the basis of the experimental data we have defined a class of peptides and derivatives or analogs thereof having the sought biological activity.

Therefore, the main object of the invention is to provide peptides having an amino acid sequence of Formula I (SEQ ID NO: 1):
X₁ X₂ X₃ X₄ PAA X₅ XXXX in which
X₁, if present, can be Aspartic acid or derivative thereof;
X₂, if present, can be Alanine or derivative thereof;
X₃, if present, can be Glycine or derivative thereof;
X₄, if present, can be Alanine or derivative thereof;
X₅ is selected between Gly and Lys; and
X, if present, is independently selected from Asp, Ala, Pro and Val as well as any derivative or analogue thereof and wherein peptides having the following sequences are excluded from Formula I: APAAG, GPAAG and Et-O—C(O)—PAAG-OMe.

Preferably, X is selected from the group consisting of Ala, Pro and Val.

According to another preferred embodiment of the invention, a peptide of the invention has an amino acid sequence selected from the group consisting of SEQ ID NO. 2, 3, 4, 5 and 6.

The expression “derivative or analogue” means any compound whose chemical structure contains little modifications with respect to the parent peptide. Such a modification has the aim to protect sites subjected to enzymatic degradation in vivo or to improve membrane penetration (such as intestinal barrier or blood-brain barrier), but it does not destroy the biological activity of the starting peptide and does not impart any toxicity. Therefore, this definition also includes those derivatives, which can be prepared from the functional groups present on the lateral chains of the amino acid moieties as well as all analogues of the parent peptide that people skilled in the art would also call “peptidomimetics”. Most of these analogues are not synthetically accessible by a chemical reaction starting from the parent peptide, but the skilled in the art knows that they can be prepared for example starting from the corresponding modified amino acids.

Therefore, according to the invention any of the above mentioned peptides may be chemically modified to be more “protected” against enzymatic degradation in vivo and more capable of penetrating membrane barriers, thus increasing its half-life and maintaining or improving its biological activity. Any chemical modification known in the art can be employed according to the present invention. We report here below some of the most common chemical modifications, which can be carried out on the chemical structure to protect peptides.

Some examples of such derivatives or analogues include compounds designed starting from the above-mentioned peptides, but showing the following chemical modifications:
1. Modifications to the N-terminal and/or C-terminal ends of the peptides: N-terminal acylation (preferably acetylation) or desamination; modification of the C-terminal carboxyl group into an amide or an alcohol group.
2. Modifications at the amide bond between two amino acids: acylation (preferably acetylation) or alkylation (preferably methylation) at the nitrogen atom or the alpha carbon of the amide bond linking two amino acids.
3. Modifications at the alpha carbon of the amide bond linking two amino acids: acylation (preferably acetylation) or alkylation (preferably methylation) at the alpha carbon of the amide bond linking two amino acids.
4. Chirality changes: replacement of one or more naturally occurring amino acids (L enantiomer) with the corresponding D-enantiomers; these kinds of modified peptides will be here indicated with the same amino acid one-letter-code, but with lower case letters (“a” means the D-enantiomer of amino acid “A” [Ala]).
5. Retro-inversion: replacement of one or more naturally-occurring amino acids (L-enantiomer) with the corresponding D-enantiomers together with an inversion of the amino acid chain (from the C-terminal end to the N-terminal end).
6. Azapeptides: replacement of one or more alpha carbons by nitrogen atoms.
7. Mixture of several modifications.

According to the invention, the preferred derivatives or analogues are those coming from the modifications of the above paragraphs 1 and 2.

“C₁-C₄-alkyl” refers to monovalent branched or unbranched alkyl groups having 1 to 4 carbon atoms. This term is exemplified by groups such as methyl, ethyl, n-propyl, isopropyl, n-butyl, isobutyl, tert-butyl and the like.

“C₁-C₃-alkyl” refers refers to monovalent branched or unbranched alkyl groups having 1 to 3 carbon atoms. This term is exemplified by groups such as methyl, ethyl, n-propyl, isopropyl and the like.

“C₂-C₄ Acyl” refers to a group —C(O)R where R includes “C₁-C₃-alkyl” groups.

The compounds of the invention can be administered as salts. Such salts include: salts of carboxyl groups or acid addition salts of amino groups of the peptide of the invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid.

As an example of peptides of the invention and of their possible derivatives or analogues, we will here report some general formulae.

A preferred embodiment of the invention includes peptides having the amino acid sequence of SEQ ID NO: 2 represented by Formula II below: 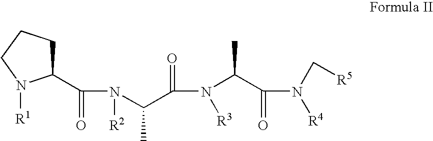
in which
R¹, R², R³ and R⁴ are hydrogen atoms and in which R⁵ is a —C(O)OH group.

However, also comprised within the present invention are those compounds of Formula II, in which:
R¹, R², R³ and R⁴ are the same or different and are selected from the group consisting of hydrogen atoms, C₂-C₄ acyl and optionally substituted C₁-C₄ alkyl groups;
R⁵ is a group selected among —C(O)N(R6)₂, —C(O)OR6, —CH₂—O—R6, wherein R⁶ are the same or different and are selected from the group consisting of hydrogen atoms and optionally substituted C₁-C₄ alkyl groups.

A preferred embodiment of the invention includes a peptide derivative of Formula II in which R¹ is acetyl, R², R³ and R⁴ are hydrogen atoms and R⁵ is —C(O)NH₂, which can be also represented as Ac-PAAG-NH₂.

Another preferred embodiment of the invention includes a peptide derivative of Formula II in which R¹ is hydrogen, R², R³ and R⁴ are methyl groups and R⁵ is —C(O)OH.

Another preferred embodiment of the invention includes peptide derivatives of Formula II in which R¹ is acetyl, at least one of R², R³ and R⁴ is a methyl group and R⁵ is —C(O)NH₂.

Other derivatives of peptides of the invention are “paag” and “gaap”.

Similarly, analogous derivatives can be designed for any of the peptides having the amino acid sequence of Formula I.

The peptides of the invention may be prepared by any well-known procedure in the art, such as solid phase synthesis or liquid phase synthesis. As a solid phase synthesis, for example, the amino acid corresponding to the C-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the C-terminus to the N-terminus, and one where the amino acids bound to the resin or the protective group of the -amino groups of the peptides are released, the peptide chain is thus extended in this manner. Solid phase synthesis methods are largely classified by the t-Boc method and the Fmoc method, depending on the type of protective group used.

Typically used protective groups include t-Boc (t-butoxycarbonyl), Cl-Z (2-chlorobenzyloxycarbonyl), Br-Z (2-bromobenzyloxycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Cl2-Bzl (2,6-dichlorobenzyl) for the amino groups; NO₂ (nitro) and Pmc (2,2,5,7,8-pentamethylchromane-6-sulphonyl) for the guanidino groups; and t-Bu (t-butyl) for the hydroxyl groups.

After synthesis of the desired peptide, it is subjected to the deprotection reaction and cut out from the solid support. Such peptide cutting reaction may be carried with hydrogen fluoride or trifluoromethane sulfonic acid for the Boc method, and with TFA for the Fmoc method.

The crude peptide thus obtained is then subjected to purification. Purification is carried out by any one of the methods known for this purpose, i.e. any conventional procedure involving extraction, precipitation, chromatography, electrophoresis, or the like. For example, HPLC (high performance liquid chromatography) can be used. The elution can be carried out using a water-acetonitrile-based solvent commonly employed for protein purification.

Any further subsequent chemical derivatization is carried out according to any method in the art.

Another object of the present invention is the use of peptides having an amino acid sequence of Formula I and derivatives thereof as a medicament.

Another object of the present invention is the use of compounds of formula II as a medicament.

Still a further object of the present invention is the use of peptides having an amino acid sequence of Formula III (SEQ ID NO: 1).

X₁X₂X₃X₄ PAA X₅ XXXX in which
X₁, if present, can be Aspartic acid or derivative thereof;
X₂, if present, can be Alanine or derivative thereof;
X₃, if present, can be Glycine or derivative thereof;
X₄, if present, can be Alanine or derivative thereof;
X₅ is selected between Gly and Lys; and
X, if present, is selected between Asp, Ala, Pro and Val as well as any derivative or analogue thereof for the preparation of a medicament useful in the treatment or prevention of transmissible spongiform encephalopathies, in particular CJD.

Just for the sake of clarity, it should be noted that the peptides having the following sequences are included in Formula III: APAAG, GPAAG and Et-O—C(O)—PAAG-OMe.

Another object of the present invention is a method for treating or preventing a Transmissible Spongiform Encephalopathy (TSE), the method comprising administering an effective dose of the above-mentioned peptides and derivatives thereof to a subject in the need thereof, wherein the subject can be human or animal.

The above-mentioned peptides and derivatives of the present invention may be administered by any means that achieves its intended purpose. For example, administration may be by a number of different routes including, but not limited to subcutaneous, intravenous, intradermal, intramuscular, intraperitoneal, intra-cerebral, intrathecal, intranasal, oral, transdermal, or buccal.

Parenteral administration can be bolus injection or by gradual perfusion over time. A typical regimen for preventing, suppressing, or treating a transmissible spongiform encephalopathy, comprises either (1) administration of an effective amount in one or two doses of a high concentration of inhibitory peptides in the range of 0.5 to 10 mg of peptide, more preferably 0.5 to 5 mg of peptide, or (2) administration of an effective amount of the peptide in multiple doses of lower concentrations of inhibitor peptides in the range of 10-1000 μg, more preferably 50-500 μg over a period of time up to and including several months to several years. It is understood that the dosage administered will be dependent upon the age, sex, health, and weight of the recipient, kind of concurrent treatment, if any, frequency of treatment, and the nature of the effect desired. The total dose required for each treatment may be administered by multiple doses or in a single dose.

By “effective amount”, it is meant a concentration of peptide(s) which is capable of slowing down or inhibiting the formation of PrP^{Sc} deposits, or of dissolving preformed deposits. Such concentrations can be routinely determined by those of skill in the art. It will also be appreciated by those of skill in the art that the dosage may be dependent on the stability of the administered peptide. A less stable peptide may require administration in multiple doses. Preparations for parenteral administration include sterile aqueous or non-aqueous solutions, suspensions, and emulsions, which may contain auxiliary agents or excipients which are known in the art. Pharmaceutical compositions such as tablets and capsules can also be prepared according to routine methods.

Pharmaceutical compositions comprising the peptides of the invention include all compositions wherein the peptide(s) are contained in an amount effective to achieve its intended purpose. In addition, the pharmaceutical compositions may contain suitable pharmaceutically acceptable carriers comprising excipients and auxiliaries which facilitate processing of the active compounds into preparations which can be used pharmaceutically. Suitable pharmaceutically acceptable vehicles are well known in the art and are described for example in Gennaro Alfonso, Ed., Remington's Pharmaceutical Sciences, 18th Edition 1990, Mack Publishing Co., Easton, Pa., a standard reference text in this field. Pharmaceutically acceptable vehicles can be routinely selected in accordance with the mode of administration and the solubility and stability of the peptides. For example, formulations for intravenous administration may include sterile aqueous solutions which may also contain buffers, diluents and other suitable additives.

Suitable formulations for parenteral administration include aqueous solutions of the active compounds in water-soluble form, for example, water-soluble salts. In addition, suspension of the active compound as appropriate oily injections suspensions may be administered.

“Pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringer's solution.

Besides the pharmaceutically acceptable carrier, the compositions of the invention can also comprise minor amounts of additives, such as stabilizers, excipients, buffers and preservatives.

The present invention has been described with reference to the specific embodiments, but the content of the description comprises all modifications and substitutions, which can be brought by a person skilled in the art without extending beyond the meaning and purpose of the claims.

The invention will now be described by means of the following Examples, which should not be construed as in any way limiting the present invention. The Examples will refer to the Figures specified here below.

### DESCRIPTION OF THE FIGURES

FIG. 1 reports the scheme of prion protein sequence used as template to design β-sheet breaker peptides.

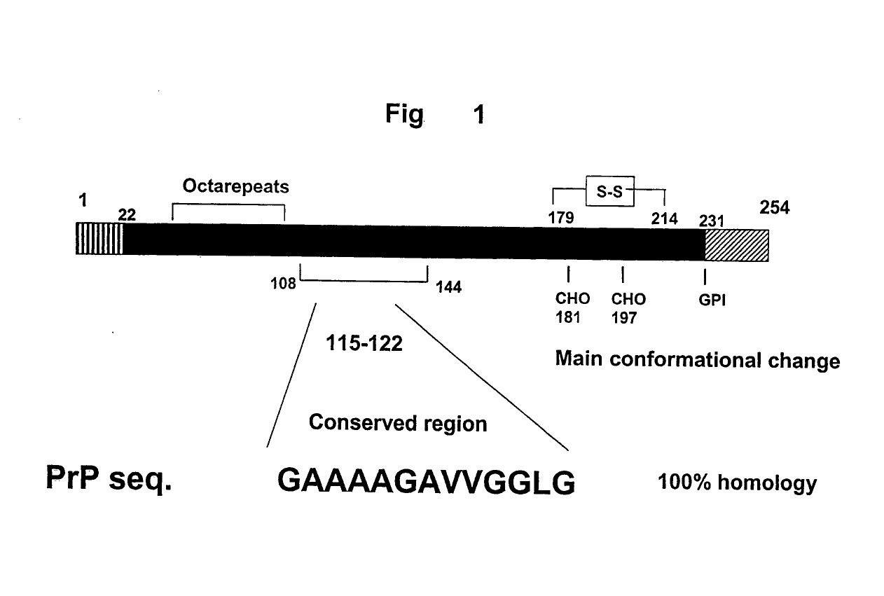

FIG. 2 shows all the peptides as well as some derivatives or analogs tested as potential prion inhibitors. “Ac” means acetylation at the N-terminus; “Am” means amidation at the C-terminus; “d” means the D-enantiomer of aspartic acid; “v” means the d-enatiomer of valine.

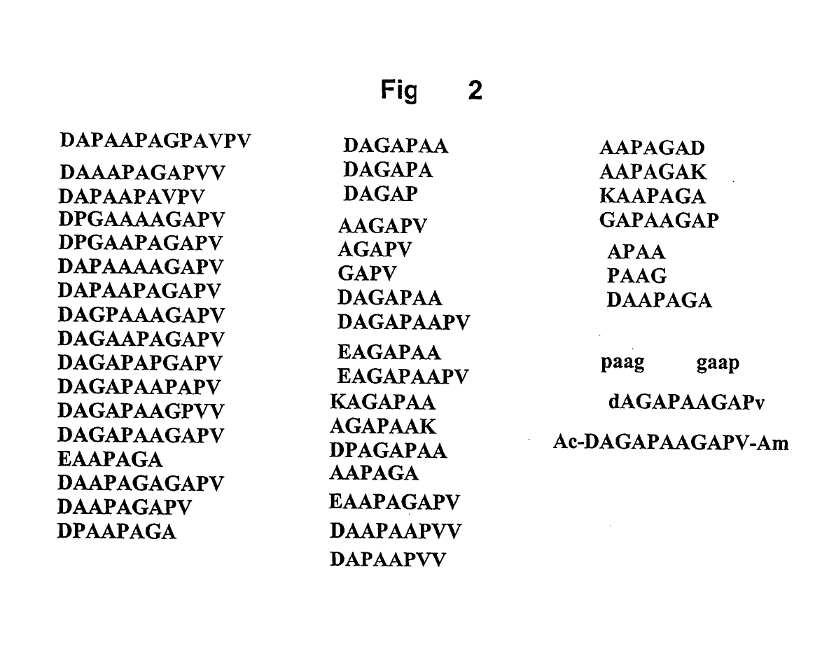

FIG. 3 reports a flow chart representing the primary screening assay for in vitro activity.

FIG. 4 is a schematic representation of the secondary cellular assay for in vitro activity.

FIG. 5 reports a table showing the in vitro activity and stability data for the set of peptides selected as active using the primary assay. “Ac” means acetylation at the N-terminus; “Am” means amidation at the C-terminus; “d” means the D-enantiomer of aspartic acid; “v” means the d-enatiomer of valine.

FIG. 6 represents the strategy used to improve stability of the 4-residue active β-sheet breaker peptide. “Ac” means acetylation, “sNMe” means a methyl group attached to the amide nitrogen, small cap letters represents the corresponding D-enantiomeric amino acids.

FIG. 7 shows a Table showing in vitro activity and stability of selected peptides and derivatives or analogs thereof.

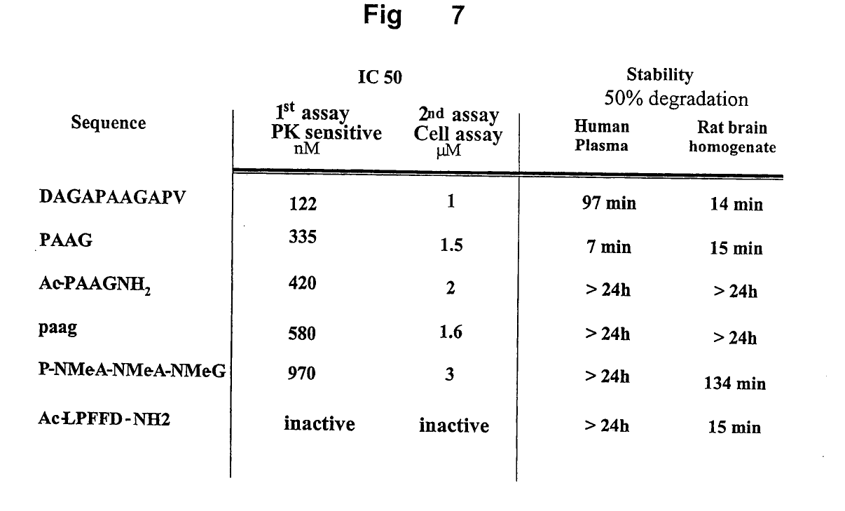

FIG. 8 shows a table showing in vivo activity of a peptide of the invention (Ac-PAAG-Am) as measured by the number of days in which clinical symptoms of scrapie appeared (incubation time) in the different dilution conditions and in presence (PK+) or absence (PK−) of Proteinase K treatment. Incubation times are given for non-treated mice (PrP^{sc} alone: groups 1 & 3) and for mice treated with a peptide of the invention (groups 2 & 4).

FIG. 9 reports the incubation times observed for each mouse in each four groups (as described for FIG. 8) of treatment for dilutions 10⁻⁴ (FIG. 9A) and 10⁻⁵ (FIG. 9B).

### EXAMPLES

### Example 1

### Peptides Synthesis

Peptides were synthesized in solid phase at Neosystem Inc. Peptides were purified by HPLC and purity (>95%) evaluated by peptide sequencing and laser desorption mass spectrometry. Stock solution of the peptides were prepared in water/0.1% trifluoroacetic acid and stored lyophilized in aliquots at −70° C. Concentration of the stock solution was estimated by amino acid analysis.

The chemical derivatization reactions were done during the synthesis at Neosystem Inc. using standard procedures.

### Example 2

### Biological Assays

In vitro assays

Two assays were used to screen for in vitro activity.

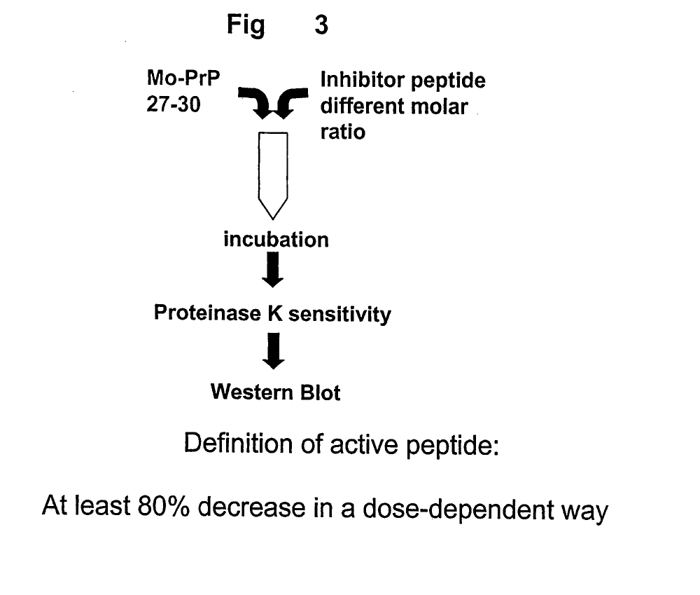

The primary screening assay consisted in incubating the abnormal form of PrP, extracted from the brains of hamsters affected by scrapie, with different concentrations of the putative inhibitors of the invention (FIG. 3).

Aliquots of 20 μl containing approximately 10 ng of partially purified PrP^{Sc} from the brain of mouse infected by 139A scrapie strain, were incubated for 2 days at 37° C. with different concentrations of peptides of the invention. After two days of incubation, samples were treated with proteinase K (PK) at a concentration of 20 μg/ml during 30 min. The PK reaction was stopped by addition of the protease inhibitor phenyl-methyl-sulfonylfluoride (PMSF) at a final concentration of 50 mM. The PK treatment permits the evaluation of the presence of the abnormal protein, since the extent of protease-resistance among PrP correlates with the pathologic and infectious features of PrP^{Sc}.

Thereafter, PrP signal was detected by western blot using 6`4 (Prionics Inc) as primary antibody and Enhanced Chemiluminescence (ECL) as detection system (Soto, et al., 2000).

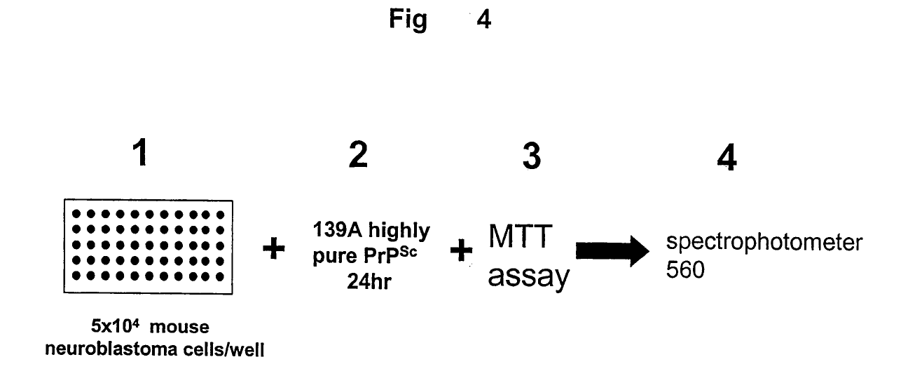

A secondary assay was used to validate compounds active in the primary assay. This was a cellular model of neuronal apoptosis induced by PrP^{Sc} (FIG. 4). The assay is done in ELISA plates and is based on the toxicity of the abnormal prion protein to neurons in culture. The IC₅₀ for the toxicity of the misfolded protein is 2.3 nM, indicating that it is highly neurotoxic, whereas the normal prion protein (PrP^{C}) is not toxic. The mouse neuroblastoma cell line N2a (American Type Culture Collection) was used for these experiments. Cells were grown using the standard conditions and were maintained at 37° C. in a humidified atmosphere with 5% CO₂. To assess cell viability, cultures were treated with mouse PrP^{Sc} pre-incubated with different concentrations of putative inhibitors of the invention for 2 days at 37° C. Cytotoxicity was evaluated by a cell proliferation assay based on reduction of 3-[4,5-dimethylthiazol-2-yl]-2,5-diphenyltetrazolium bromide (MTT) following the instructions from the manufacturer (Boehringer Mannheim).

Stability of Peptides of the Invention

The peptides of the invention were prepared as a 1 μg/μl solution in water.

20 μl of the peptide solution was diluted in 80 μl of fresh human plasma or 80 μl of 10% rat brain homogenate. The solution was incubated at 37° C. for different time periods and the reaction was stopped by adding a complete <<cocktail>> of protease inhibitors. The bulk of the plasma proteins (none of the peptide) were precipitated in cold methanol (mix/MeOH, ⅘, v/v) for one hour at −20° C. The precipitated proteins were pelleted by centrifugation (10,000 rpm, 10 min, 4° C.). The supernatant, containing the peptide, was concentrated 5 times under vacuum and separated by reverse-phase HPLC. The peak area corresponding to the intact peptide was measured and compared with an equivalent sample incubated without plasma.

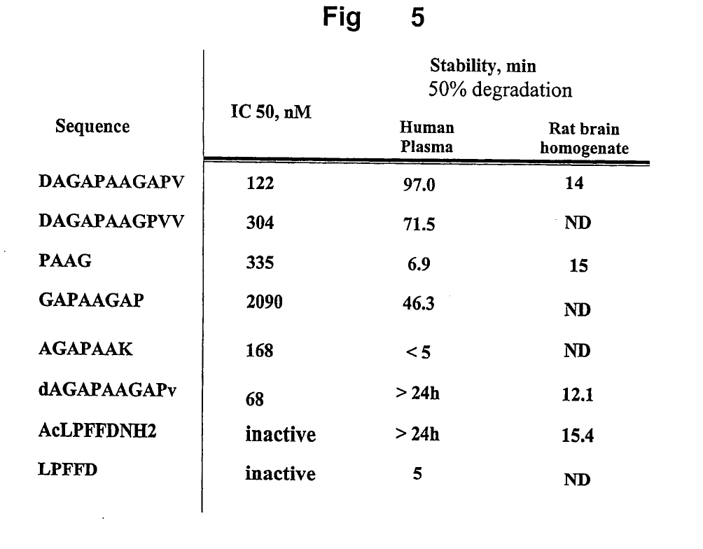

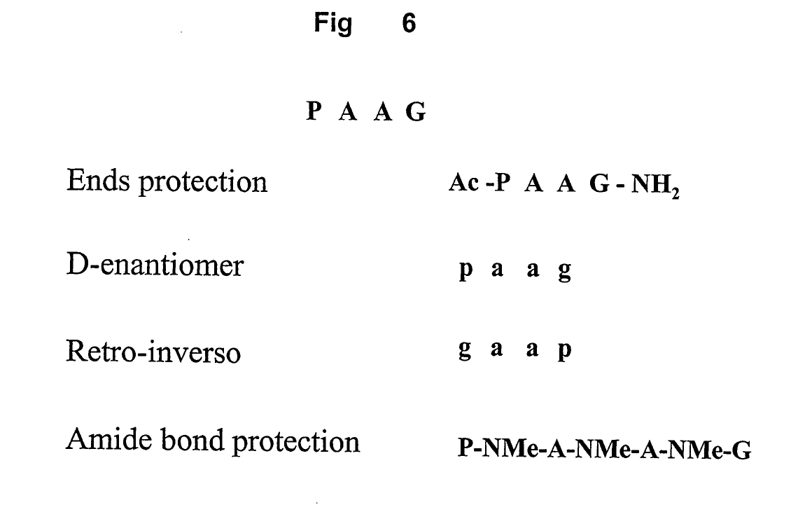

Peptides of the invention were able to reverse the protease-resistance of abnormal PrP by at least 80%. The IC₅₀ were calculated as well as the time in which 50% of the peptide was degraded in human plasma and rat brain homogenate and the results are shown in FIG. 5 for 6 peptides of the invention. The 4-residue-long peptide having SEQ ID NO: 2 (PAAG) is particularly interesting, because the small size make it more amenable to chemical modifications and has higher possibilities to penetrate membrane barriers, such as the blood-brain barrier. A weakness of this peptide is its short stability (FIG. 5), so we evaluated several strategies to minimize peptide degradation (FIG. 6). These strategies included: (a) end-protection by acetylation at the N-terminus and amidation at the C-terminus (Ac-PAAG-Am); (b) synthesizing the peptide in all D-amino acids (paag); (c) the retro-inverse version of the peptide (gaap); (d) the same sequence containing N-methylations in all 3 peptide bonds. Among these derivatives at least two of them (Ac-PAAG-Am and paag) showed a similar activity in the primary and secondary assay while stability was dramatically increased.

In Vivo Assay:

The in vivo effect of Ac-PAAG-Am was studied in mice with experimental scrapie, which is regarded as a model for prion diseases (Kimberlin, 1976). The experiments were carried out with the mouse-adapted scrapie strain 139A, following a protocol similar to our previous study (Soto et al., 2000). The level of scrapie infectivity in the presence or in the absence of the compound of the invention was measured by incubation time assays using different dilutions of the infectious material.

PrP^{sc} was purified from mice infected with 139A scrapie strain as previously described (Soto et al., 2000). Briefly, brain tissue was solubilized in 20% sarkosyl and subjected to differential centrifugation employing a Beckman TL100 ultracentrifuge. Final pellets were re-suspended in Tris buffered saline containing 0.1% SB-314. After this procedure PrP^{sc} represented 50-60% of total protein, as evaluated by SDS-PAGE and silver staining.

The partially purified protein was incubated with Ac-PAAG-Am at a 1:1000 molar ratio (PrP^{sc}: peptide) during 48 h at 37° C. Half of the sample was thereafter treated with proteinase K. Several dilutions of these samples were used for inoculation into animals.

C57BL/6J mice were divided in four groups for inoculation. The mice were inoculated intra-cerebrally with 25 μl of brain extract (treated or untreated) using a 0.5 ml insulin syringe with a 28-gauge needle inserted into the right parietal. The material to be injected was diluted at three different dilutions (10⁻³, 10⁻⁴ and 10⁻⁵) for each group of treatment. Ten mice per group of treatment are used, i.e. a total of 140 mice (0.20 controls inoculated with PBS buffer).

The four groups of mice were injected with the following treatments:
Group 1 PrP^{Sc} (infectious agent) alone
Group 2 PrP^{Sc}+Ac-PAAG-Am
Group 3 PrP^{Sc} alone digested with proteinase K (PK)
Group 4 (PrP^{Sc}+Ac-PAAG-Am) digested with proteinase K (PK)

Animals in group 1 were injected with partly purified PrP^{Sc} incubated for 2 days alone at 37° C.

Group 2 animals were inoculated with the same sample incubated for 2 days with Ac-PAAG-Am (0.15 μg/μl).

Groups 3 and 4 were the same as 1 and 2 respectively, but after incubation without or with the peptide of the invention (Ac-PAAG-Am), the sample was treated with proteinase K (PK) for 30 min at 37° C. at a protease concentration of 50 μg/ml. The purpose of PK treatment was to remove Ac-PAAG-Am together with the fraction of the pathogenic isoform (PrP^{sc}) which has been reverted into normal conformation of the protein (PrP^{c}) after the peptide addition.

Begining 13 weeks after inoculation the onset of clinical disease was measured by observing mice twice a week on a grid system as previously described (Carp et al., 1984) and measuring body weight. Scrapie incubation periods were determined from the date of injection to the time mice exhibit signs of clinical disease for 3 consecutive weeks. Increase in incubation time is indicative of alteration in the level of scrapie infectivity. Results were statistically analyzed by unpaired t-test and Mann Whitney test with significance accepted at the P<0.05 level.

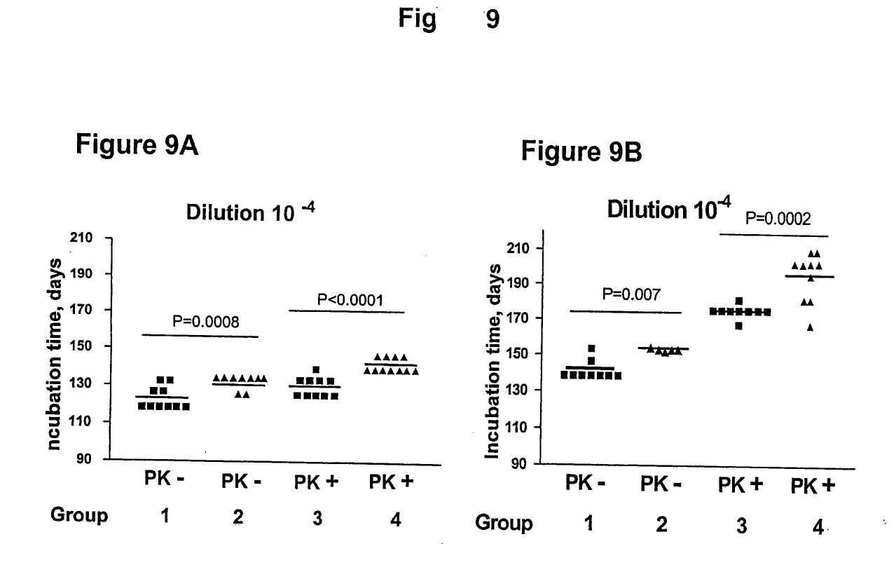

Animals infected with brain extracts treated with Ac-PAAG-Am (groups 2 and 4) developed scrapie symptoms significantly later than mice inoculated with PrP^{Sc} incubated alone (FIGS. 18 and 9).

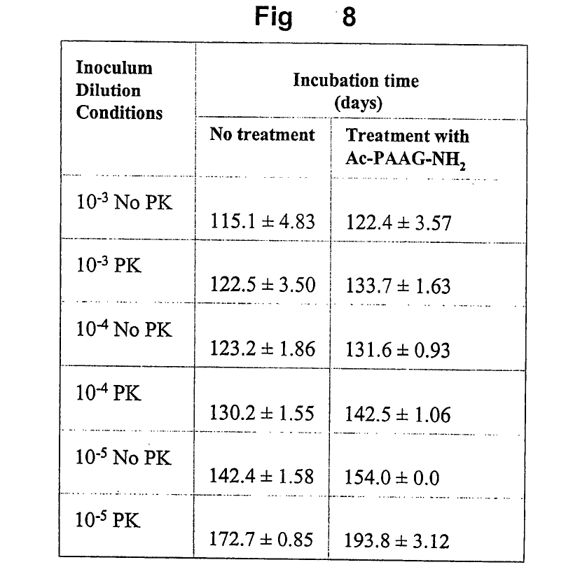

As expected, the efficacy of the compound of the invention depends on the concentration of the infectious inoculum (PrP^{Sc}), at molar ratio PrP^{sc}: peptide constant. In animals inoculated at 10⁻³, no really significant difference was observed between groups 1 (no treatment) and 2 (treatment) (FIG. 8), while at lower concentration of the inoculum, a clear effect on retardation of the onset of the disease is observed for the group containing the peptide of the invention (group 2) compared to group 1 (FIGS. 9A and 9B).

### REFERENCES

Baldwin et al., J. Biol. Chem. 270, 19197-19200, 1995;
Bruce et al., nature, 389, 498-501, 1997;
Carp, R. I., Callahan, S. M., Sersen, E. A., and Moretz, R. C. Intervirology 21, 61-69, 1984;
Cohen et al., Science 264, 530-531, 1994;
Cohen et al., Ann. Rev. Biochem. 67, 793-819, 1998;
Cousens et al., Nature, 385, 197-198, 1997;
Kimberlin, R. H. Science Progress 63, 461-481, 1976;
Pan et al., Proc. Natl. Acad. Sci. (USA) 90, 10962-10966, 1993;
Prusiner, Science 252, 1515-1522, 1991;
Roos et al., Brain 96, 1-20, 1973;
Soto et al., Biohem. Biophys. Res. Commun., 226(3), 672-680, Sep. 24, 1996;
Soto et al., Nature Med., 4(7), 822-826, July 1998;
Soto et al., Lancet, 355, 192-197, Jan. 15, 2000; and
Stahl at al., Biochem. 32, 1991-2002, 1993.

1. A peptide comprising an amino acid sequence of Formula I (SEQ ID NO: 1): X₁ X₂ X₃ X₄ PAA X₅ XXXX, wherein each of X₁, X₂, X₃, X₄ and one or more X is optionally present, and wherein
X₁, if present, is Aspartic acid or derivative thereof;
X₂, if present, is Alanine or derivative thereof;
X₃, if present, is Glycine or derivative thereof;
X₄, if present, is Alanine or derivative thereof;
X₅ is Gly or Lys; and
X, if present, is independently selected from Asp, Ala, Pro, Val, any derivative thereof or analogue thereof, and
wherein sequences: APAAG, GPAAG and Et-O—C(O)—PAAG-O—Me are excluded from formula I;
or a derivative or analogue of said peptide.

2. The peptide according to claim 1, wherein X, when present, is selected from Ala, Pro or Val.

3. The peptide according to claim 1, wherein the peptide has an amino acid sequence selected from SEQ ID NO: 2, SEQ ID NO: 3, SEQ ID NO: 4, SEQ ID NO: 5 or SEQ ID NO: 6.

4. The peptide according to claim 1, represented by the compound of Formula II below: 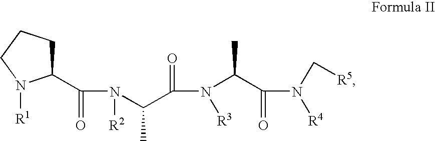
wherein
R¹, R², R³ and R⁴ are the same or different, and are selected from the group consisting of hydrogen, C₂-C₄ acyl and C₁-C₄ alkyl;
R⁵ is selected from the group consisting of CON(R⁶)₂, COOR⁶ and CH₂OR⁶, wherein
R⁶ is selected from the group consisting of hydrogen and C₁-C₄ alkyl.

5. The compound according to claim 4, wherein R¹ is acetyl, and R², R³ and R⁴ are hydrogen and R⁵ is —C(O)NH₂.

6. The compound according to claim 4, wherein R¹ is H, and R², R³ and R⁴ are methyl groups and R⁵ is —C(O)OH.

7. A medicament comprising an effective amount of the peptide according to claim 1, and a suitable carrier.

8. A method of preparing a medicament for treating or preventing transmissible spongiform encephalopathies, comprising adding to a suitable carrier a peptide comprising an amino acid sequence of formula III (SEQ ID NO: 1): X₁ X₂ X₃ X₄ PAA X₅ XXXX, wherein each of X₁, X₂, X₃, X₄ and one or more X is optionally present, and wherein
X₁, if present, is Aspartic acid or derivative thereof;
X₂, if present, is Alanine or derivative thereof;
X₃, if present, is Glycine or derivative thereof;
X⁴, if present, is Alanine or derivative thereof;
X₅ Gly or Lys; and
X, if present, is selected from Asp, Ala, Pro, Val, Gly, any derivative thereof or analogue thereof
or a derivative or analogue of said peptide.

9. The method according to claim 8, wherein the transmissible spongiform encephalopathy is CJD.

10. A pharmaceutical composition for the treatment or prevention of transmissible spongiform encephalopathies, comprising an effective amount of the peptide of claim 1, as active ingredient, and a pharmaceutically acceptable excipient.

11. A method of treating or preventing a transmissible spongiform encephalopathy, comprising administering an effective amount of the peptide of claim 1 to a subject in the need thereof.

12. The method according to claim 11, in which the subject is human.

13. A medicament comprising an effective amount of the compound according to claim 4 and a suitable carrier.

14. A compound according to Formula II below: 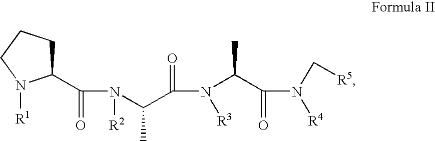
wherein
R¹, R², R³ and R⁴ are the same or different, and are selected from the group consisting of hydrogen, C₂-C₄ acyl and C₁-C₄ alkyl;
R⁵ is selected from the group consisting of CON(R⁶)₂, COOR⁶ and CH₂OR⁶, wherein R⁶ is hydrogen or C₁-C₄ alkyl.
